(19) United States
(12) Patent Application Publication
(10) Pub. No.: US 2006/0189545 A1
(43) Pub. Date: Aug. 24, 2006

(54) Novel chemical entities and methods for their use in treatment of metabolic disorders

(76) Inventors: Samuel T. Henderson, Broomfield, CO (US); Steve Orndorff, Arvada, CO (US); Lawrence Melvin, Longmont, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/546,976

(22) PCT Filed: Mar. 8, 2004
(86) PCT No.: PCT/US04/07191

§ 371 Date: Aug. 25, 2005

Related U.S. Application Data
(60) Provisional application No. 60/452,855, filed on Mar. 6, 2003.

(51) Int. Cl. A61K 31/7024; C07H 13/06

(52) U.S. Cl. 514/23; 514/25; 536/119

(57) ABSTRACT

Methods and composition for treating or preventing, the occurrence of senile dementia of the Alzheimer's type, or other conditions arising from reduced neuronal metabolism and leading to lessened cognitive function are described. In a preferred embodiment the administration of novel esterified saccharide compounds to said patient at a level to produce an improvement in cognitive ability.

### FIELD OF THE INVENTION

This invention relates to the field of therapeutic agents for the treatment of Alzheimer's disease, and other diseases associated with reduced neuronal metabolism, including Parkinson's disease, Huntington's Disease, and epilepsy. The therapeutic agents are esterified saccharides, many of them novel compounds.

### BACKGROUND OF THE INVENTION

Alzheimer's disease (AD) is a progressive neurodegenerative disorder, which primarily affects the elderly. There are two forms of AD, early-onset and late-onset. Early-onset AD is rare, strikes susceptible individuals as early as the third decade, and is frequently associated with mutations in a small set of genes. Late onset, or spontaneous, AD is common, strikes in the seventh or eighth decade, and is a mutifactorial disease with many genetic risk factors. Late-onset AD is the leading cause of dementia in persons over the age of 65. An estimated 7-10% of the American population over 65, and up to 40% of the American population greater than 80 years of age is afflicted with AD (McKhann et al., 1984; Evans et al. 1989). Early in the disease, patients experience loss of memory and orientation. As the disease progresses, additional cognitive functions become impaired, until the patient is completely incapacitated. Many theories have been proposed to describe the chain of events that give rise to AD, yet, at the time of this application, the cause remains unknown. Currently, no effective prevention or treatment exists for AD. The only drugs to treat AD on the market today, Aricept®, Cognex®, Reminyl® and Exelon® are acetylcholinesterase inhibitors. These drugs do not address the underlying pathology of AD. They merely enhance the effectiveness of those nerve cells still able to function and only provide symptomatic relief from the disease. Since the disease continues, the benefits of these treatments are slight.

Metabolism and Alzheimer's Disease. At the time of this application, the cause of AD remains unknown, yet a large body of evidence has made it clear that Alzheimer's disease is associated with decreased neuronal metabolism. In 1984, Blass and Zemcov proposed that AD results from a decreased metabolic rate in sub-populations of cholinergic neurons. However, it has become clear that AD is not restricted to cholinergic systems, but involves many types of transmitter systems, and several discrete brain regions. Positron-emission tomography has revealed poor glucose utilization in the brains of AD patients, and this disturbed metabolism can be detected well before clinical signs of dementia occur (Reiman et al., 1996; Messier and Gagnon, 1996; Hoyer, 1998). Additionally, certain populations of cells, such as somatostatin cells of the cortex in the AD brain are smaller, and have reduced Golgi apparatus; both indicating decreased metabolic activity (for review see Swaab et al. 1998). Measurements of the cerebral metabolic rates in healthy versus AD patients demonstrated a 20-40% reduction in glucose metabolism in AD patients (Hoyer, 1992). Reduced glucose metabolism results in critically low levels of ATP in AD patients. Also, the severity of decreased metabolism was found to correlate with senile plaque density (Meier-Ruge, et al. 1994).

Additionally, molecular components of insulin signaling and glucose utilization are impaired in AD patients. Glucose is transported across the blood brain barrier and is used as a major fuel source in the adult brain. Consistent with the high level of glucose utilization, the brains of mammals are well supplied with receptors for insulin and IGF, especially in the areas of the cortex and hippocampus, which are important for learning and memory (Frolich et al., 1998). In patients diagnosed with AD, increased densities of insulin receptor were observed in many brain regions, yet the level of tyrosine kinase activity that normally is associated with the insulin receptor was decreased, both relative to age-matched controls (Frolich et al., 1998). The increased density of receptors represents up-regulation of receptor levels to compensate for decreased receptor activity. Activation of the insulin receptor is known to stimulate phosphatidylinositol-3 kinase (PI3K). PI3K activity is reduced in AD patients (Jolles et al., 1992; Zubenko et al., 1999). Furthermore, the density of the major glucose transporters in the brain, GLUT1 and GLUT3 were found to be 50% of age matched controls (Simpson and Davies, 1994). The disturbed glucose metabolism in AD has led to the suggestion that AD may be a form of insulin resistance in the brain, similar to type II diabetes (Hoyer, 1998). Inhibition of insulin receptor activity can be exogenously induced in the brains of rats by intracerebroventricular injection of streptozotocin, a known inhibitor of the insulin receptor. These animals develop progressive defects in learning and memory (Lannert and Hoyer, 1998). While glucose utilization is impaired in brains of AD patients, use of the ketone bodies, beta-hydroxybutyrate and acetoacetate may be unaffected (Ogawa et al., 1996).

The cause of decreased neuronal metabolism in AD remains unknown. Yet, aging may exacerbate the decreased glucose metabolism in AD. Insulin stimulation of glucose uptake is impaired in the elderly, leading to decreased insulin action and increased insulin resistance (for review see Finch and Cohen, 1997). For example, after a glucose load, mean plasma glucose is 10-30% higher in those over 65 than in younger subjects. Hence, genetic risk factors for AD may result in slightly compromised neuronal metabolism in the brain. These defects would only become apparent later in life when glucose metabolism becomes impaired, and thereby contribute to the development of AD. Since the defects in glucose utilization are limited to the brain in AD, the liver does not mobilize fatty acids (see Brain Metabolism section below). Without ketone bodies to use as an energy source, the neurons of the AD patient brain slowly starve to death.

Attempts to compensate for reduced cerebral metabolic rates in AD patients has met with some success. Treatment of AD patients with high doses of glucose and insulin increases cognitive scores (Craft et al., 1996). However, since insulin is a polypeptide and must be transported across the blood brain barrier, delivery to the brain is complicated. Therefore, insulin is administered systemically. A large dose of insulin in the blood stream can lead to hyperinsulinemia, which will cause irregularities in other tissues. Both of these shortcomings make this type of therapy difficult and rife with complications. Accordingly, there remains a need for an agent that may increase the cerebral metabolic rate and subsequently the cognitive abilities of a patient suffering from Alzheimer's disease.

Brain Metabolism. The brain has a very high metabolic rate. For example, it uses 20 percent of the total oxygen consumed in a resting state. Large amounts of ATP are required by neurons of the brain for general cellular functions, maintenance of an electric potential, synthesis of neurotransmitters and synaptic remodeling. Current models propose that under normal physiologic conditions, neurons of the adult human brain depend solely on glucose for energy. Since neurons lack glycogen stores, the brain depends on a continuous supply of glucose from the blood for proper function. Hence, sudden interruption of glucose delivery to the brain results in neuronal damage. Yet, if glucose levels drop gradually, such as during fasting, neurons will begin to metabolize ketone bodies instead of glucose and no neuronal damage will occur.

Neuronal support cells, glial cells, are much more metabolically diverse and can metabolize many substrates, in particular, glial cells are able to utilize fatty acids for cellular respiration. Neurons of the brain cannot efficiently oxidize fatty acids and hence rely on other cells, such as liver cells and astrocytes to oxidize fatty acids and produce ketone bodies. Ketone bodies are produced from the incomplete oxidation of fatty acids and are used to distribute energy throughout the body when glucose levels are low. In a normal Western diet, rich in carbohydrates, insulin levels are high and fatty acids are not utilized for fuel, hence blood ketone body levels are very low, and fat is stored and not used. Current models propose that only during special states, such as neonatal development and periods of starvation, will the brain utilize ketone bodies for fuel. The partial oxidation of fatty acids gives rise to D-3-hydroxybutyrate (D-β-hydroxybutyrate) and acetoacetate, which together with acetone are collectively called ketone bodies. Neonatal mammals are dependent upon milk for development. The major carbon source in milk is fat (carbohydrates make up less then 12% of the caloric content of milk). The fatty acids in milk are oxidized to give rise to ketone bodies, which then diffuse into the blood to provide an energy source for development. Numerous studies have shown that the preferred substrates for respiration in the developing mammalian neonatal brain are ketone bodies. Consistent with this observation is the biochemical finding that astrocytes, oligodendrocytes and neurons all have capacity for efficient ketone body metabolism (for review see Edmond, 1992). Yet only astrocytes are capable of efficient oxidation of fatty acids to ketone bodies.

The body normally produces small amounts of ketone bodies. However, because they are rapidly utilized, the concentration of ketone bodies in the blood is very low. Blood ketone body concentrations rise on a low carbohydrate diet, during periods of fasting, and in diabetics. In a low carbohydrate diet, blood glucose levels are low, and pancreatic insulin secretion is not stimulated. This triggers the oxidation of fatty acids for use as a fuel source when glucose is limiting. Similarly, during fasting or starvation, liver glycogen stores are quickly depleted, and fat is mobilized in the form of ketone bodies. Since both a low carbohydrate diet and fasting do not result in a rapid drop of blood glucose levels, the body has time to increase blood ketone levels. The rise in blood ketone bodies provides the brain with an alternative fuel source, and no cellular damage occurs. Since the brain has such high energy demands, the liver oxidizes large amounts of fatty acids until the body becomes literally saturated with ketone bodies. Therefore, when an insufficient source of ketone bodies is coupled with poor glucose utilization severe damage to neurons results. Since glial cells are able to utilize a large variety of substrates they are less susceptible to defects in glucose metabolism than are neurons. This is consistent with the observation that glial cells do not degenerate and die in AD (Mattson, 1998).

As discussed in the Metabolism and Alzheimer's disease section, in AD, neurons of the brain are unable to utilize glucose and begin to starve to death. Since the defects are limited to the brain and peripheral glucose metabolism is normal, the body does not increase production of ketone bodies, therefore neurons of the brain slowly starve to death. Accordingly, there remains a need for an energy source for brain cells that exhibit compromised glucose metabolism. Compromised glucose metabolism is a hallmark of AD; hence administration of an alternative energy source will prove beneficial to those suffering from AD.

Huntington's Disease

Huntington's disease (HD) is a familial neurodegenerative disorder that afflicts 1/10,000 individuals. It is inherited in an autosomal dominant manner and is characterized by choreiform movements, dementia, and cognitive decline. The disease is produced by genes containing a variably increased (expanded) CAG repeat within the coding region. The size range of the repeats is similar in all diseases; unaffected individuals have fewer than 30 CAG repeats, whereas affected patients usually have more than 40 repeats. The disorder usually has a mid-life onset, between the ages of 30 to 50 years, but may in some cases begin very early or much later in life. The size of the inherited CAG repeat correlates with the severity and age of disease onset. The CAG triplet repeat produces a polyglutamine domain in the expressed proteins. The symptoms are progressive and death typically ensues 10 to 20 years after onset, most often as the result of secondary complications of the movement disorder.

The mutant gene produces huntingtin protein, whose function is unknown. The polyglutamine regions of Huntingtin interact with glyceraldehyde-3-phosphate dehydrogenase (GAPDH), a key glycolytic enzyme. While normal glutamine can bind with GAPDH and cause no harm to the enzyme, binding of mutant Huntingtin inhibits the enzyme. It is believed that the lack of energy being supplied to the brain cells, due to the interference of the Huntingtin protein with GAPDH, in part, causes neuron damage in the basal ganglia and the cerebral cortex. Mitochondrial dysfunction has also been implicated HD.

At least four other diseases are caused by the expanded CAG repeat, and thus also may implicate defective glucose metabolism. These include spinobulbar muscular atrophy, dentatorubral-pallidoluysian atrophy (DRPLA), spino-cerebellar ataxia type 1, and spino-cerebellar ataxia type 3.

Parkinson's Disease

Parkinson's disease (PD) is widely considered to be the result of degradation of the pre-synaptic dopaminergic neurons in the brain, with a subsequent decrease in the amount of the neurotransmitter dopamine that is being released. Inadequate dopamine release, therefore, leads to the onset of voluntary muscle control disturbances symptomatic of PD.

The motor dysfunction symptoms of PD have been treated in the past using dopamine receptor agonists, monoamine oxidase binding inhibitors, tricyclic antidepressants, anticholinergics, and histamine H1-antagonists. Unfortunately, the main pathologic event, degeneration of the cells in substantia nigra, is not helped by such treatments. The disease continues to progress and, frequently after a certain length of time, dopamine replacement treatment will lose its effectiveness. In addition to motor dysfunction, however, PD is also characterized by neuropsychiatric disorders or symptoms. These include apathy-amotivation, depression, and dementia. PD patients with dementia have been reported to respond less well to standard L-dopa therapy. Moreover, these treatments have little or no benefit with respect to the neuropsychiatric symptoms. Impaired neuronal metabolism is believed to be a contributing factor to PD.

Epilepsy

Epilepsy, sometimes called a seizure disorder, is a chronic medical condition produced by temporary changes in the electrical function of the brain, causing seizures which affect awareness, movement, or sensation. There has been long experience with ketogenic diets, which mimic starvation, in children treated for epilepsy. The diet is a medical therapy and should be used under the careful supervision of a physician and/or dietician. The diet carefully controls caloric input and requires that the child eat only what has been included in the calculations to provide 90% of the day's calories as fats. However, such diets are generally unsuitable for use in adults due to: (1) adverse effects on the circulatory system from incorporation of long chain triglycerides as the primary fat in these diets into cholesterol and the effects of hyperlipidemia; (2) poor patient compliance due to the unappealing nature of the low carbohydrate diet.

There remains a need, therefore, for therapeutic agents for diseases of impaired metabolism.

Co-pending U.S. patent application Ser. No. 10/152,147, filed May 20, 2002, entitled “Use of Medium Chain Trigylcerides for the Treatment and Prevention of Alzheimer's Disease and Other Diseases Resulting from Reduced Neuronal Metabolism II,” and Ser. No. 09/845,741, filed May 1, 2001, entitled “Use of Medium Chain Trigylcerides for the Treatment and Prevention of Alzheimer's Disease and Other Diseases Resulting from Reduced Neuronal Metabolism” describe methods of treating or preventing dementia of the Alzheimer's type, or other loss of cognitive function caused by reduced neuronal metabolism, comprising administering an effective amount of medium chain triglycerides to a patient in need thereof. The applications demonstrate that medium chain triglycerides (MCT) and their associated fatty acids are useful as a treatment and preventative measure for AD patients and other diseases and conditions resulting from reduced neuronal metabolism. The applications show that ingestion of MCTs leads to increased levels of blood ketone bodies and thereby provide energy to starving brain neurons, thereby restoring neuronal metabolism.

The present invention provides therapeutic agents, many of which are novel compounds, which, like MCTs, will lead to increased levels of blood ketone bodies and restore neuronal metabolism upon ingestion. Compounds similar to those described herein have been utilized previously, in other applications, for example, in cosmetic applications (WO 00/61079) and as excipients in foodstuffs (WO 91/15963), but not therapeutic applications.

### SUMMARY OF THE INVENTION

The present invention provides a compound of the formula: 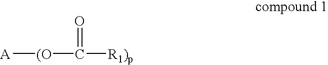
wherein A represents a saccharide moiety, p is the number of free hydroxyl groups on the saccharide moiety A, and R₁ is independently selected from a fatty acid residue having 5-12 carbons in the carbon backbone (C5 to C12 fatty acids) esterified to the saccharide, a saturated fatty acid residue having 5-12 carbons in the carbon backbone (C5 to C12 fatty acids) esterified to the saccharide, an unsaturated fatty acid residue having 5-12 carbons in the carbon backbone (C5 to C12 fatty acids) esterified to the saccharide, and derivatives of any of the foregoing. In one embodiment, the compound is not described in Takada, et al., 1991 nor Jandacek & Webb, 1978. In one embodiment, R₁ comprises C₈ fatty acid residues. In another embodiment the compound has the structure

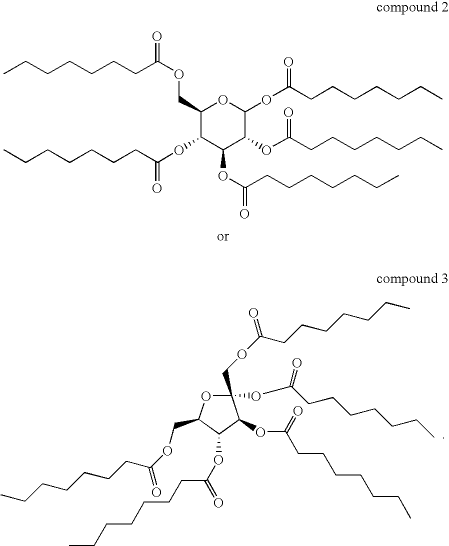

The present invention also provides a compound of the formula: 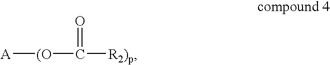
wherein R₂ is independently selected from the group consisting of R₁, an essential fatty acid esterified to the saccharide, β-hydroxybutyrate esterified to the saccharide, acetoacetate esterified to the saccharide, compound 5 esterified to the saccharide, and compound 6 esterified to the saccharide. In one embodiment, the compound is not described in Takada, et al., 1991 nor Jandacek & Webb, 1978. In one embodiment, R₂ is either acetoacetate esterified to the saccharide or β-hydroxybutyrate esterified to the saccharide. In another embodiment, the ratio of β-hydroxybutyrate R₂ groups to acetoacetate R₂ groups is about 3:2 to 4:1, with a ratio of 3:1 being preferable. In another embodiment, the invention provides a mixture of a first compound and a second compound, wherein the first compound R₂ group is β-hydroxybutyrate; and the second compound R₂ group is acetoacetate, and wherein the first compound and the second compound are present in a 3:2 to 4:1 ratio, with a ratio of 3:1 being preferable.

The present invention also provides a pharmaceutical composition, comprising a TCA cycle intermediate and a compound of the formulas; 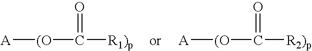
described above. In one embodiment, the TCA cycle intermediate is selected from a group consisting of citric acid, aconitic acid, isocitric acid, α-ketoglutaric acid, succinic acid, fumaric acid, malic acid, oxaloacetic acid, and mixtures thereof.

In another embodiment, the present invention also provides a pharmaceutical composition, comprising a precursor of a TCA cycle intermediate and a compound of the formulas: 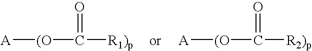
described above. In some embodiments, the precursor of a TCA cycle intermediate is a compound which, upon administration to a human being, is converted in vivo to form a TCA cycle intermediate. In other embodiments, the precursor is selected from a group consisting of 2-keto-4-hydroxypropanol, 2,4-dihydroxybutanol, 2-keto-4-hydroxybutanol, 2,4-dihydroxybutyric acid, 2-keto-4-hydroxybutyric acid, aspartates, mono- and di-alkyl oxaloacetates, pyruvate, and glucose-6-phosphate.

In another embodiment, the present invention also provides a pharmaceutical composition, comprising a ketone body or metabolic precursor of a ketone body and a compound of the formulas: 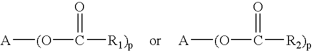
described above. In one embodiment, the ketone body or metabolic precursor is selected from a group consisting of β-hydroxybutyrate, acetoacetate, metabolic precursors of β-hydroxybutyrate or acetoacetate, and mixtures thereof. In other embodiments, the metabolic precursor is a physiologically acceptable salt or ester of a polymer or oligomers wherein in each case the number of subunit repeats is selected such that the polymer or oligomers is readily metabolized on administration to a human or animal to provide elevated ketone body levels in the blood. In still further embodiments, the metabolic precursor is selected from the group consisting of: 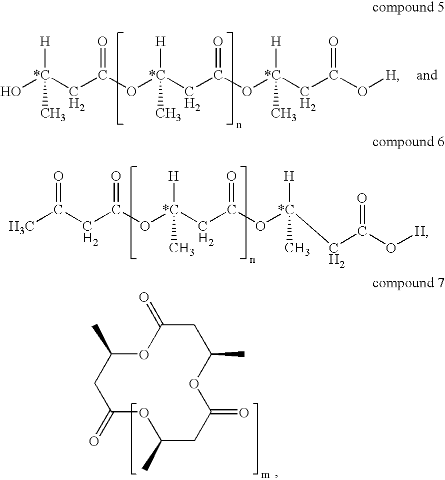
wherein n is an integer of 0 to 1,000, and m is an integer of 1 or more, a complex thereof with one or more cations or a salt thereof for use in therapy or nutrition.

The present invention further provides a pharmaceutical composition comprising a metabolic adjuvant and a compound a selected from the group consisting of a compound of the formula and a compound of the formulas: 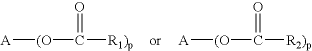
described above. In one embodiment, the adjuvant is selected from a group consisting of a vitamin, a mineral, an antioxidant, an energy-enhancing compound, and mixtures thereof. In another embodiment, the energy-enhancing compound is selected from a group consisting of Coenzyme CoQ-10, creatine, L-carnitine, n-acetyl-carnitine, L-carnitine derivatives, and mixtures thereof. In other embodiments, the vitamin is selected from a group consisting of ascorbic acid, biotin, calcitriol, cobalamin, folic acid, niacin, pantothenic acid, pyridoxine, retinol, retinal (retinaldehyde), retinoic acid, riboflavin, thiamin, α-tocopherol, phytylmenaquinone, multiprenylmenaquinone, pyridoxine derivatives, pantothenic acid, and mixtures thereof. In still other embodiments, the mineral is selected from a group consisting of calcium, magnesium, sodium, potassium, zinc, copper, aluminum, chromium, vanadium, selenium, phosphorous, manganese, iron, fluorine, cobalt, molybdenum, iodine and mixtures thereof. In still other embodiments, the antioxidant is selected from a group consisting of ascorbic acid, α-tocopherol, and mixtures thereof.

The present invention further provides a pharmaceutical composition comprising a therapeutic agent selected from the group consisting of acetylcholinesterase inhibitors, acetylcholine synthesis modulators, acetylcholine storage modulators, acetylcholine release modulators, anti-inflammatory agents, estrogen or estrogen derivatives, insulin sensitizing agents, β-amyloid plaque removal agents (including vaccines), inhibitors of β-amyloid plaque formation, γ-secretase modulators, pyruvate dehydrogenase complex modulators, α-ketoglutarate dehydrogenase complex modulators, neurotrophic growth factors (e.g., BDNF), ceramides or ceramide analogs, and NMDA glutamate receptor antagonists; and a compound of the formulas: 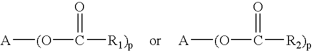
described above.

The present invention also provides a pharmaceutical composition comprising at least one therapeutic agent that induces utilization of fatty acids and a compound of the formulas: 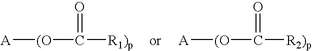
described above. In one embodiment, the therapeutic agent which induces utilization of fatty acids is selected from the group consisting of a PPAR-gamma agonist, a statin drug, and a fibrate drug. In further embodiments, the PPAR-gamma agonist is selected from the group consisting of aspirin, ibuprofen, ketoprofen, and naproxen, and thiazolidinedione drugs. In still further embodiments, the statin drug is Lipitor or Zocor. In still further embodiments, the fibrate drug is selected from the group consisting of Bezafibrate, ciprofibrate, fenofibrate and Gemfibrozil. In still further embodiments, the therapeutic agent is caffeine and ephedra.

The present invention also provides a method of elevating ketone body levels comprising administering a compound of the formulas: 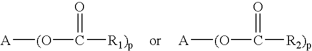
described above.

The present invention also provides a method of increasing cognitive ability in a patient suffering from Alzheimer's disease, comprising administering a compound of the formulas: 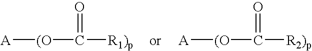
described above. In some embodiments, the increase in cognitive ability is measured by a test selected from the group consisting of ADAS-cog, MMSE, Stroop Color Word Interference Task, Logical Memory subtest of the Wechsler Memory Scale-III, Clinician's Dementia Rating, and Clinician's Interview Based Impression of Change.

The present invention further provides a method of increasing cognitive ability in a patient suffering from Alzheimer's disease, comprising increasing ketone body levels in said patient, said increasing accomplished by the administration of a compound of the formulas: 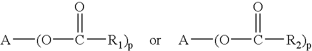
described above. In some embodiments, the increase in cognitive ability is measured by a test selected from the group consisting of ADAS-cog, MMSE, Stroop Color Word Interference Task, Logical Memory subtest of the Wechsler Memory Scale-III, Clinician's Dementia Rating, and Clinician's Interview Based Impression of Change.

The present invention further provides a method of treating or preventing dementia of Alzheimer's type, or other loss of cognitive function caused by reduced neuronal metabolism, comprising administering an effective amount of a compound selected from the group consisting of a compound of the formulas: 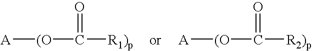
described above. In some embodiments, the compound is administered at a dose of about 0.01 g/kg/day to about 10 g/kg/day.

### BRIEF DESCRIPTION OF THE FIGURES

FIG. 1 shows a schematic of the TCA cycle as it occurs in the cell.

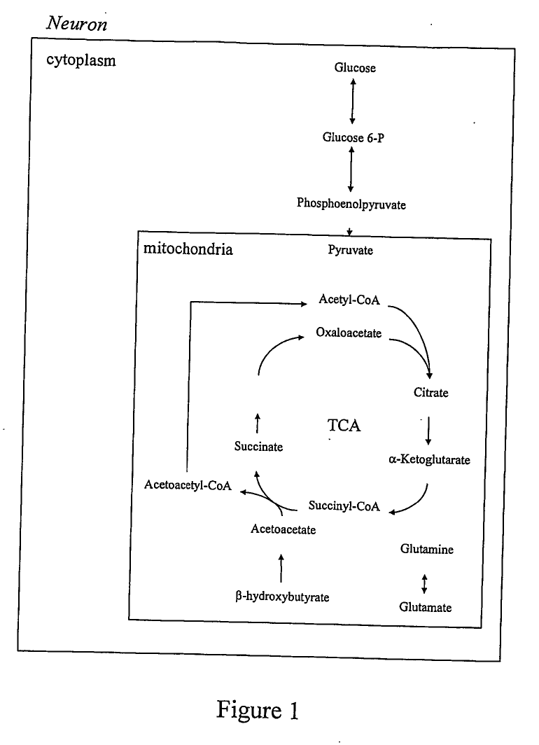

FIG. 2 shows the results of treatment on cognitive performance for apoE4+ and apoE4− patients for treatment with medium chain triglycerides.

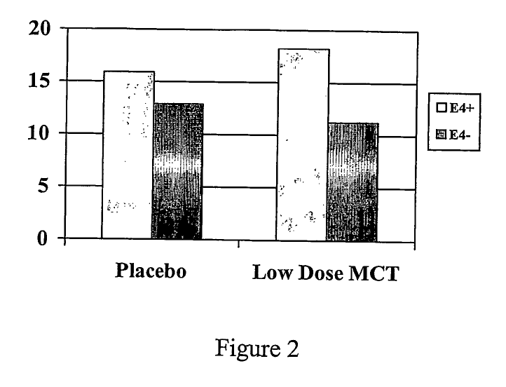

### DETAILED DESCRIPTION OF THE INVENTION

The present invention relates to esterified saccharide compounds and compositions suitable for administration to humans and animals which have the properties of, inter alia, (i) increasing cardiac efficiency, particularly efficiency in use of glucose, (ii) providing a source of energy, particularly in diabetes and insulin resistant states and (iii) treating disorders caused by damage to brain cells, particularly by retarding or preventing brain damage in memory associated brain areas such as found in Alzheimer's and similar conditions.

As described in the Background section, neurons of the brain can use both glucose and ketone bodies for respiration. The neurons of Alzheimer's disease patients have well documented defects in glucose metabolism, and known genetic risk factors for Alzheimer's disease are associated with lipid and cholesterol transport, suggesting defects in triglyceride usage that may underlie susceptibility to Alzheimer's disease. It is therefore an object of this invention to provide novel chemical entities which, upon ingestion will lead to increased levels of blood ketone bodies and thereby provide energy to starving brain neurons. Additionally, defects in neuronal metabolism in Huntington's disease, Parkinson's disease, and epilepsy and other related neurodegenerative diseases such as Wernicke-Korsakoff disease and possibly schizophrenia will be benefited by high blood ketone levels, derived from therapeutic agents that provide an energy source for brain cells. As used herein, “high blood ketone levels” refers to levels of at least about 0.1 mM. More preferably, high blood ketone levels refers to levels in the range of 0.1 to 50 mM, more preferably in the range of 0.2-20 mM, more preferably in the range of 0.3-5 mM, and more preferably in the range of 0.5-2 mM.

The esterified saccharide compounds of the present invention will be administered in a dosage required to increase blood ketone bodies to a level required to treat and prevent the occurrence of Alzheimer's disease. Ketone bodies are produced by the oxidation of fatty acids in tissues that are capable of such oxidation. The major organ for fatty acid oxidation is the liver. Under normal physiological conditions, ketone bodies are rapidly utilized and cleared from the blood. Under some conditions, such as starvation or low carbohydrate diet, ketone bodies are produced in excess and accumulate in the blood stream. Compounds that mimic the effect of increasing oxidation of fatty acids will raise ketone body concentration to a level to provide an alternative energy source for neuronal cells with compromised metabolism. Since the efficacy of such compounds derives from their ability to increase fatty acid utilization and raise blood ketone body concentration they are dependent on the embodiments of the present invention.

Compounds that mimic the effect of increasing oxidation of fatty acids and will raise ketone body concentration include but are not limited to the ketone bodies, D-β-hydroxybutyrate and aceotoacetate, and metabolic precursors of these. The term metabolic precursor, as used herein, refers to compounds that comprise 1,3 butane diol, acetoacetyl or D-β-hydroxybutyrate moieties such as acetoacetyl-1-1,3-butane diol, acetoacetyl-D-β-hydroxybutyate, and acetoacetylglycerol. Esters of any such compounds with monohydric, dihydric or trihydric alcohols is also envisaged. Metabolic precursors also include polyesters of D-β-hydroxybutyrate, and acetoaoacetate esters of D-β-hydroxybutyrate. Polyesters of D-β-hydroxybutyrate include oligomers of this polymer designed to be readily digestible and/or metabolized by humans or animals. These preferably are of 2 to 100 repeats long, typically 2 to 20 repeats long, and most conveniently from 3 to 10 repeats long. Examples of poly D-β-hydroxybutyrate or terminally oxidized poly-D-β-hydroxybutyrate esters useable as ketone body precursors are given below:

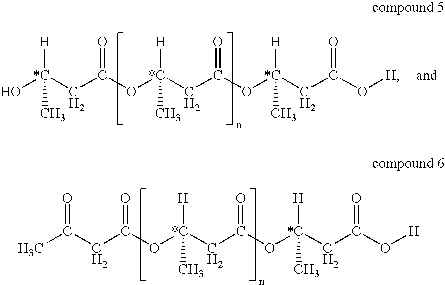

In each case n is selected such that the polymer or oligomer is readily metabolized on administration to a human or animal body to provide elevated ketone body levels in blood. Preferred values of n are integers of 0 to 1,000, more preferably 0 to 200, still more preferably 1 to 50, most preferably 1 to 20, particularly conveniently being from 3 to 5. Examples of cations and typical physiological salts are described herein, and additionally include sodium, potassium, magnesium, calcium, each balanced by a physiological counter-ion forming a salt complex, L-lysine, L-arginine, methyl glucamine, and others known to those skilled in the art. The preparation and use of such metabolic precursors is detailed in Veech, WO 98/41201, and Veech, WO 00/15216, each of which is incorporated by reference herein in its entirety.

Accordingly, the present invention is directed toward esterified saccharide compounds of the formula

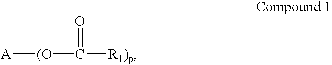

wherein A represents a saccharide moiety, p is the number of free hydroxyl groups on the saccharide moiety A, and R₁ is independently selected from a fatty acid residue having 5-12 carbons in the carbon backbone (C5 to C12 fatty acids) esterified to the saccharide, a saturated fatty acid residue having 5-12 carbons in the carbon backbone (C5 to C12 fatty acids) esterified to the saccharide, an unsaturated fatty acid residue having 5-12 carbons in the carbon backbone (C5 to C12 fatty acids) esterified to the saccharide, and derivatives of any of the foregoing. With regard to p, for example, if A is fructofuranose or glucopyranose, then p is 5. If A is cellobiose or maltose, then n is 8.

Without being bound by theory, it is believed that the ester linkage will be easily hydrolyzed in vivo to provide short chain fatty acids, which will be totally metabolized to ketone bodies.

Saccharide, as used herein, refers to a group of water-soluble carbohydrates of relatively low molecular weight. The simple sugars are called monosaccharides. More complex sugars comprise between two and ten monosaccharides linked together: disaccharides contain two, trisaccharides three, and so on. Saccharides include but are not limited to L- and D-isomers and α- and β-forms where appropriate, including monosaccharides such as glucose, fructose, mannose, streptose, an aldose including aldomonose, aldodiose, aldotriose, aldotetrose, aldopentose, aldohexose, aldoheptose, aldooctose, aldononose, and aldodecose, a ketose including ketomionose, ketodiose, ketotriose, ketotetrose, ketopentose, ketohexose, ketoheptose, ketooctose, ketononose, and ketodecose, idose, galactose, allose, arabinose, gulose, fucose, glycose, glycosulose, erythrose, threose, ribose, xylose, lyxose, altrose, idose, talose, erythrulose, ribulose, mycarose, xylulose, psicose, sorbose, tagatose, acid, glucaric acid, gluconic acid, glucuronic acid, glyceraldehyde, glucopyranose, glucofuranose, aldehydoglucose, arabinofuranose, galacturonic acid, manuronic acid, glucosamine, galactosamine and neuraminic acid, disaccharides such as sucrose, maltose, cellobiose, lactose, strophanthobiose, and trehalose, and trisaccharides such as maltotriose, raffinose, cellotriose or manninotriose.

Esterified, as used herein, refers to a linkage between a saccharide hydroxyl (—OH) group and the acid portion (COO—) of the fatty acid or other acid to form a typical ester bond (ROOR′).

Certain of these compounds have been described previously. Takada, et al., 1991 describes the preparation and thermal properties of cellobiose octa(n-alkanoates). Jandacek & Webb, 1978 describe the preparation and physical properties of pure sucrose octaesters. Neither Takada, et al., 1991 nor Jandacek & Webb, 1978 even remotely suggests that the compounds can be used for therapeutic purposes. Indeed, neither article indicates any possible use for these compounds. The compounds described in Takada, et al., 1991 nor Jandacek & Webb, 1978 are specifically excluded from this invention.

Preferred embodiments of Compound 1 include, but are not limited to α-D-glucopyranose pentaoctanoate:

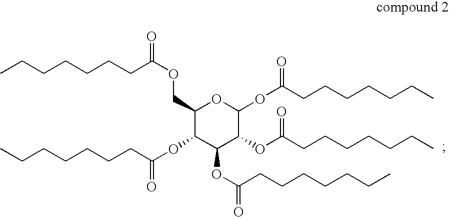

β-D-fructose pentaoctanoic acid ester:

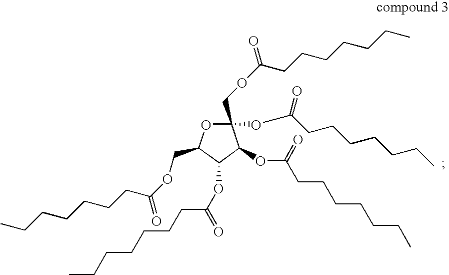

and maltose octanoic acid ester:

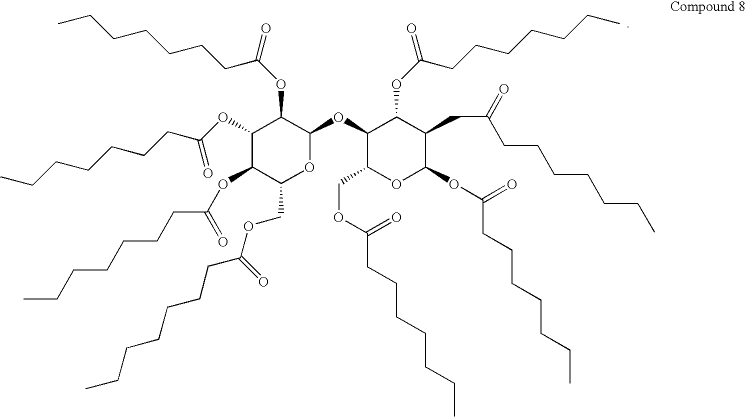

The present invention is also directed toward esterified saccharide compounds of the formula:

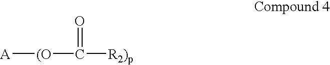

wherein R₂ is independently selected from the group consisting of R₁, an essential fatty acid esterified to the saccharide, β-hydroxybutyrate esterified to the saccharide, acetoacetate esterified to the saccharide, compound 5 esterified to the saccharide, and compound 6 esterified to the saccharide. This compound will provide increased levels of ketone bodies due to the character of the molecule where R₂ is a ketone body precursor of the molecule. Additionally, where R₂ is an essential fatty acid, namely, linoleic or arachidonic acids, the compound has the additional advantage of providing the essential fatty acid.

Preferred compounds of the formula Compound 4 include, but are not limited to compounds wherein R₂ is either acetoacetate esterified to the saccharide or β-hydroxybutyrate esterified to the saccharide; compounds wherein R₂ is either acetoacetate esterified to the saccharide or β-hydroxybutyrate esterified to the saccharide, and wherein the ratio of β-hydroxybutyrate R₂ groups to acetoacetate R₂ groups is about 3:2 to 4:1, with a ratio of 3:1 being preferable, as not all sugars have a number of free hydroxyl groups that make a 3:1 ratio possible. For example, for compounds wherein A is a fructofuranose, the β-hydroxybutyrate to acetoacetate R₂ group ratio may be 4:1 or 3:2. Alternatively, mixtures of compounds wherein A is a fructofuranose and wherein R₂ is β-hydroxybutyrate; and wherein A is a fructofuranose and wherein R₂ is acetoacetate may be prepared in a 3:1 ratio.

Another preferred compound includes

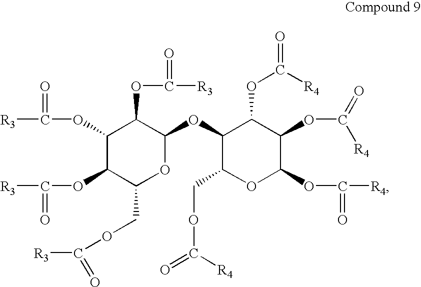

wherein three of R₃ are esters of octanoic acid, and the fourth of R₃ is an ester of acetoacetate, and wherein three of R₄ are esters of octanoic acid, and the fourth of R₄ is an ester of acetoacetate.

The present invention provides a method of treating or preventing dementia of Alzheimer's type, or other loss of cognitive function caused by reduced neuronal metabolism, comprising administering an effective amount of a esterified saccharide compound of the formula compound 1 and/or compound 4 to a patient in need thereof. Generally, an effective amount is an amount effective to either (1) reduce the symptoms of the disease sought to be treated or (2) induce a pharmacological change relevant to treating the disease sought to be treated. For Alzheimer's disease, an effective amount includes an amount effective to: increase cognitive scores; slow the progression of dementia; or increase the life expectancy of the affected patient.

The esterified saccharide compounds of the invention may be prepared by any method known in the art, including the methods of Takada, et al., 1991, and Jandacek & Webb, 1978.

In a preferred embodiment, the method comprises the use of compound 1 wherein R₁ is a fatty acid containing an eight-carbon backbone.

In another preferred embodiment, the invention comprises the coadministration of compound 1 and/or compound 4 and L-carnitine or a derivative of L-carnitine. Slight increases in MCFA oxidation have been noted when MCTs are combined with L-carnitine (Odle, 1997). Thus in the present invention compound 1 and/or compound 4 are combined with L-carnitine at doses required to increase the utilization of said compound 1 and/or compound 4. The dosage of L-carnitine and compound 1 and/or compound 4 will vary according to the condition of the host, method of delivery, and other factors known to those skilled in the art, and will be of sufficient quantity to raise blood ketone levels to a degree required to treat and prevent Alzheimer's Disease. Derivatives of L-carnitine which may be used in the present invention include but are not limited to decanoylcarnitine, hexanoylcarnitine, caproylcarnitine, lauroylcarnitine, octanoylcarnitine, stearoylcarnitine, myristoylcarnitine, acetyl-L-carnitine, O-Acetyl-L-carnitine, and palmitoyl-L-carnitine.

Therapeutically effective amounts of the therapeutic agents can be any amount or dose sufficient to bring about the desired anti-dementia effect and depend, in part, on the severity and stage of the condition, the size and condition of the patient, as well as other factors readily known to those skilled in the art. The dosages can be given as a single dose, or as several doses, for example, divided over the course of several weeks.

In one embodiment, compound 1 and/or compound 4 are administered orally. In another embodiment, compound 1 and/or compound 4 are administered intravenously. Oral administration of MCT and preparations of intravenous MCT solutions are well known to those skilled in the art, and thus provide guidance for administration and preparation of the esterified saccharide compounds of the present invention.

This invention also provides a therapeutic agent comprising for the treatment or prevention of dementia of Alzheimer's type, or other loss of cognitive function caused by reduced neuronal metabolism, comprising medium chain triglycerides. In a preferred embodiment, the therapeutic agent is provided in administratively convenient formulations of the compositions including dosage units incorporated into a variety of containers. Dosages of the esterified saccharides are preferably administered in an effective amount, in order to produce ketone body concentrations sufficient to increase the cognitive ability of patients afflicted with AD or other states of reduced neuronal metabolism. For example, for the ketone body, D-β-hydroxybutyrate, blood levels are desirably raised to about 0.1-50 mM (measured by urinary excretion in the range of about 5 mg/dL to about 160 mg/dL), more preferably raised to about 0.2-20 mM, more preferably raised to about 0.3-5 mM, more preferably raised to about 0.5-2 mM, although variations will necessarily occur depending on the formulation and host, for example. Effective amount dosages of esterified saccharides of the present invention will be apparent to those skilled in the art. In one embodiment, an esterified saccharide dose will be in the range of 0.05 g/kg/day to 10 g/kg/day of esterified saccharide. More preferably, the dose will be in the range of 0.25 g/kg/day to 5 g/kg/day of esterified saccharide. More preferably, the dose will be in the range of 0.5 g/kg/day to 2 g/kg/day of esterified saccharide. Convenient unit dosage containers and/or formulations include tablets, capsules, lozenges, troches, hard candies, nutritional bars, nutritional drinks, metered sprays, creams, and suppositories, among others. The compositions may be combined with a pharmaceutically acceptable excipient such as gelatin, an oil, and/or other pharmaceutically active agent(s). For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof, (see e.g. Goodman et al. 1996).

In a preferred embodiment, the invention provides a formulation comprising a mixture of an esterified saccharide of the present invention and carnitine to provide elevated blood ketone levels. The nature of such formulations will depend on the duration and route of administration. Such formulations will be in the range of 0.05 g/kg/day to 10 g/kg/day of esterified saccharide and 0.05 mg/kg/day to 10 mg/kg/day of carnitine or its derivatives. In one embodiment, an esterified saccharide dose will be in the range of 0.05 g/kg/day to 10 g/kg/day. More preferably, the dose will be in the range of 0.25 g/kg/day to 5 g/kg/day of esterified saccharide. More preferably, the dose will be in the range of 0.5 g/kg/day to 2 g/kg/day of esterified saccharide. In some embodiments, a carnitine or carnitine derivative dose will be in the range of 0.05 g/kg/day to 10 g/kg/day. More preferably, the carnitine or carnitine derivative dose will be in the range of 0.1 g/kg/day to 5 g/kg/day. More preferably, the carnitine or carnitine derivative dose will be in the range of 0.5 g/kg/day to 1 g/kg/day. Variations will necessarily occur depending on the formulation and/or host, for example.

In another embodiment, the invention provides a therapeutic compound or mixture of compounds, the composition and dosage of which is influenced by the patients' genotype, in particular the alleles of apoliproprotein E gene. In Example 3 of co-pending U.S. patent application Ser. No. 10/152,147, filed May 20, 2002, entitled “Use of Medium Chain Trigylcerides for the Treatment and Prevention of Alzheimer's Disease and Other Diseases Resulting from Reduced Neuronal Metabolism II,” discloses that non-E4 carriers performed better than those with the E4 allele when elevated ketone body levels were induced with MCT. Also, those with the E4 allele had higher fasting ketone body levels and the levels continued to rise at the two hour time interval. Therefore, E4 carriers may require higher ketone levels or agents that increase the ability to use the ketone bodies that are present. Accordingly, a preferred embodiment consists of a dose of an esterified saccharide of the present invention combined with agents that increase the utilization of fats, MCT or ketone bodies. Examples of agents that increase utilization of fatty acids may be selected from a group comprising of, but not limited to, non-steroidal anti-inflammatory agents NSAIDs), statin drugs (such as Lipitor® and Zocor®) and fibrates. Examples of NSAIDs include: aspirin, ibuprofen (Advil, Nuprin, and others), ketoprofen (Orudis KT, Actron), and naproxen (Aleve).

NSAIDs function, in part, as PPAR-gamma agonists. Increasing PPAR-gamma activity increases the expression of genes associated with fatty acid metabolism such as FATP (for review see (Gelman, Fruchart et al. 1999)). Accordingly, a combination of an esterified saccharide of the present invention and PPAR-gamma agonists will prove beneficial to individuals with decreased neuronal metabolism. In a preferred embodiment the PPAR-gamma agonist is an NSAID.

Statins are a class of drugs with pleiotropic effects, the best characterized being inhibition of the enzyme 3-hydroxy-3-methylglutaryl CoA reductase, a key rate step in cholesterol synthesis. Statins also have other physiologic affects such as vasodilatory, anti-thrombotic, antioxidant, anti-proliferative, anti-inflammatory and plaque stabilizing properties. Additionally, statins cause a reduction in circulating triglyceride rich lipoproteins by increasing the levels of lipoprotein lipase while also decreasing apolipoprotein C-III (an inhibitor of lipoprotein lipase) (Schoonjans, Peinado-Onsurbe et al. 1999). Accordingly, administration of statins results in increased fatty acid usage, which can act synergistically with administration of esterified saccharides of the present invention. This should prove especially beneficial to ApoE4 carriers. One embodiment of this invention would be combination therapy consisting of statins and an esterified saccharide of the present invention.

Fibrates, such as Bezafibrate, ciprofibrate, fenofibrate and Gemfibrozil, are a class of lipid lowering drugs. They act as PPAR-alpha agonists and similar to statins they increase lipoprotein lipase, apoAI and apoAII transcription and reduce levels of apoCIII (Staels, Dallongeville et al. 1998). As such they have a major impact on levels of triglyceride rich lipoproteins in the plasma, presumably by increasing the use of fatty acids by peripheral tissues. Accordingly, the present invention discloses that fibrates alone or in combination with an esterified saccharide of the present invention would prove beneficial to patients with reduced neuronal metabolism such as those with Alzheimer's disease.

Caffeine and ephedra alkaloids are commonly used in over the counter dietary supplements. Ephedra alkaloids are commonly derived from plant sources such as ma-huang (Ephedra sinica). The combination of caffeine and ephedra stimulate the use of fat. Ephedra alkaloids are similar in structure to adrenaline and activate beta-adenergic receptors on cell surfaces. These adenergic receptors signal through cyclic AMP (cAMP) to increase the use of fatty acids. cAMP is normally degraded by phosphodiesterase activity. One of the functions of caffeine is to inhibit phosphodiesterase activity and thereby increase cAMP mediated signaling. Therefore caffeine potentiates the activity of the ephedra alkaloids. Accordingly, the present invention discloses that ephedra alkaloids alone can provide a treatment or prevention for conditions of reduced neuronal metabolism. Additionally, it is disclosed that ephedra alkaloids in combination with caffeine can provide a treatment or prevention for conditions of reduced neuronal metabolism. Accordingly, it is disclosed that a combination of an esterified saccharide of the present invention with ephedra, or an esterified saccharide of the present invention with caffeine, or an esterified saccharide of the present invention, ephedra alkaloids and caffeine together can provide a treatment or prevention for conditions of reduced neuronal metabolism.

Ketone bodies are used by neurons as a source of Acetyl-CoA. Acetyl-CoA is combined with oxaloacetate to form citrate in the Krebs' cycle, or citric acid cycle (TCA cycle). It is important for neurons to have a source of Acetyl-CoA as well as TCA cycle intermediates to maintain efficient energy metabolism. Yet, neurons lose TCA cycle intermediates to synthesis reactions, such as the formation of glutamate. Neurons also lack pyruvate carboxylase and malate dehydrogenase enzymes so they cannot replenish TCA cycle intermediates from pyruvate (Hertz, Yu et al. 2000). Accordingly, the present invention discloses that a combination of ketone bodies with a source of TCA cycle intermediates will be beneficial to conditions of reduced neuronal metabolism. TCA cycle intermediates are selected from a group consisting of citric acid, aconitic acid, isocitric acid, α-ketoglutaric acid, succinic acid, fumaric acid, malic acid, oxaloacetic acid, and mixtures thereof. One embodiment of the invention is a combination of TCA cycle intermediates with an esterified saccharide of the present invention in a formulation to increase efficiency of the TCA.

Another source of TCA cycle intermediates are compounds that are converted to TCA cycle intermediates within the body (TCA intermediate precursors). Examples of such compounds are 2-keto-4-hydroxypropanol, 2,4-dihydroxybutanol, 2-keto-4-hydroxybutanol, 2,4-dihydroxybutyric acid, 2-keto-4-hydroxybutyric acid, aspartates as well as mono- and di-alkyl oxaloacetates, pyruvate and glucose-6-phosphate. Accordingly, the present invention discloses that a combination of TCA intermediate precursors with ketone bodies will be beneficial for the treatment and prevention of diseases resulting from reduced metabolism. Also, the present invention discloses that an esterified saccharide of the present invention combined with TCA intermediate precursors will be beneficial for the treatment and prevention of diseases resulting from reduced metabolism.

The present invention further discloses that additional sources of TCA cycle intermediates and Acetyl-CoA can be advantageously combined with ketone body therapy. Sources of TCA cycle intermediates and Acetyl-CoA include mono- and di-saccharides as well as triglycerides of various chain lengths and structures.

Further benefit can be derived from formulation of a pharmaceutical composition that includes metabolic adjuvants. Metabolic adjuvants include vitamins, minerals, antioxidants and other related compounds. Such compounds may be chosen from a list that includes but is not limited to; ascorbic acid, biotin, calcitriol, cobalamin, folic acid, niacin, pantothenic acid, pyridoxine, retinol, retinal (retinaldehyde), retinoic acid, riboflavin, thiamin, α-tocopherol, phytylmenaquinone, multiprenylmenaquinone, calcium, magnesium, sodium, aluminum, zinc, potassium, chromium, vanadium, selenium, phosphorous, manganese, iron, fluorine, copper, cobalt, molybdenum, iodine. Accordingly a combination of ingredients chosen from: metabolic adjuvants, compounds that increase ketone body levels, and TCA cycle intermediates, will prove beneficial for treatment and prevention of diseases associated with decreased metabolism, including Alzheimer's disease, Parkinson's disease, Huntington's disease, and epilepsy.

With regard to epilepsy, the prior art provides descriptions of ketogenic diets in which fat is high and carbohydrates are limited. In summary, the rationale of such diets is that intake of high amounts of fat, whether long-chain or medium-chain triglycerides, can increase blood ketone levels in the context of a highly-regimented diet in which carbohydrate levels are absent or limited. Limitation of carbohydrate and insulin are believed to prevent re-esterification in adipose tissue. In contrast to the prior art, the present invention provides for and claims the administration of compounds which can increase blood ketone levels outside of the context of the ketogenic diet.

Although the ketogenic diet has been known for decades, there does not appear to be any prior art teaching or suggesting that medium chain triglyceride therapy or other ketone body precursors be used to treat Alzheimer's disease or other cognitive disorders outside the metabolic constraints of the ketogenic diet.

Additional metabolic adjuvants include energy enhancing compounds, such as Coenzyme CoQ-10, creatine, L-carnitine, n-acetyl-carnitine, L-carnitine derivatives, and mixtures thereof. These compounds enhance energy production by a variety of means. Carnitine will increase the metabolism of fatty acids. CoQ10 serves as an electron carrier during electron transport within the mitochondria. Accordingly, addition of such compounds with MCT will increase metabolic efficiency especially in individuals who may be nutritionally deprived.

Administration of MCT, and especially triglycerides composed of C6 and C8 fatty acid residues, result in elevated ketone body levels even if large amounts of carbohydrate are consumed at the same time (for overview see (Odle 1997)). The advantages of the Applicant's approach are clear, since careful monitoring of what is eaten is not required and compliance is much simpler.

Further benefit can be derived from formulation of a pharmaceutical composition comprising an esterified saccharide of the present invention and other therapeutic agents which are used in the treatment of Alzheimer's disease, Parkinson's disease, Huntington's disease, or epilepsy. Such therapeutic agents include acetylcholinesterase inhibitors, acetylcholine synthesis modulators, acetylcholine storage modulators, acetylcholine release modulators, anti-inflammatory agents, estrogen or estrogen derivatives, insulin sensitizing agents, β-amyloid plaque removal agents (including vaccines), inhibitors of β-amyloid plaque formation, γ-secretase modulators, pyruvate dehydrogenase complex modulators, neurotrophic growth factors (e.g., BDNF), ceramides or ceramide analogs, and/or NMDA glutamate receptor antagonists for overview of such treatments see (Selkoe 2001; Bullock 2002)). While such treatments are still in the experimental stage it is the novel insight of the present invention that said treatments be advantageously combined with increased fatty acid/ketone body usage as described herein.

Ketone bodies can also provide a therapeutic approach to the treatment of insulin resistance where the normal insulin signaling pathway is disordered and in conditions where the efficiency of cardiac hydraulic work is decreased for metabolic reasons. It has been suggested that use of ketone bodies has great advantage over use of insulin itself. Abnormal elevation of blood sugar occurs not only in insulin deficient and non insulin dependent diabetes but also in a variety of other diseases. The hyperglycaemia of diabetes results from an inability to metabolize and the over production of glucose. Both types of diabetes are treated with diet; Type I diabetes almost always requires additional insulin, whereas non-insulin dependent diabetes, Type II diabetes, such as senile onset diabetes, may be treated with diet and weight loss, although insulin is increasingly used to control hyperglycaemia. It has been suggested that supplementing type II diabetics with ketone bodies would allow better control of blood sugar, thus preventing the vascular changes in eye and kidney which occur now after 20 years of diabetes and which are the major cause of morbidity and mortality in diabetics. Thus, the present invention provides a method of treating a human or animal in order to treat an insulin resistant state comprising administering to that person an esterified saccharide of the present invention. By insulin resistant state herein is included forms of diabetes, particularly those that do not respond fully to insulin.

Advantages

From the description above, a number of advantages of the invention for treating and preventing Alzheimer's disease become evident:
(a) Prior art on AD has largely focused on prevention and clearance of amyloid deposits. The role of these amyloid deposits in AD remains controversial and may only be a marker for some other pathology. The present invention provides a novel route for treatment and prevention of AD based on alleviating the reduced neuronal metabolism associated with AD, and not with aspects of amyloid accumulation.
(b) Current treatments for AD are merely palliative and do not address the reduced neuronal metabolism associated with AD. Ingestion of novel ketone body precursors as a nutritional supplement or therapeutic is a simple method to provide neuronal cells, in which glucose metabolism is compromised, with ketone bodies as a metabolic substrate.
(c) Levels of ketone bodies can be easily measured in urine or blood by commercially available products (e.g., Ketostix®, Bayer, Inc.).

Accordingly, the reader will see that the use of esterified saccharides of the present invention as a treatment and preventative measure of Alzheimer's disease (AD) provides a novel means of alleviating reduced neuronal metabolism associated with AD. It is the novel and significant insight of the present invention that use of these compounds will result in hyperketonemia which will provide increased neuronal metabolism for diseases associated with reduced neuronal metabolism, such as AD, ALS, Parkinson's disease and Huntington's disease. Although the description above contains many specificities, these should not be construed as limiting the scope of the invention but merely as providing illustrations for some of the presently preferred embodiments of this invention. For example, supplementation with an esterified saccharide of the present invention may prove more effective when combined with insulin sensitizing agents such as vanadyl sulfate, chromium picolinate, and vitamin E. Such agents may function to increase glucose utilization in compromised neurons and work synergistically with hyperketonemia. In another example an esterified saccharide of the present invention can be combined with compounds that increase the rates of fatty acid utilization such as L-carnitine and its derivatives. Mixtures of such compounds may synergistically increase levels of circulating ketone bodies.

Thus the scope of the invention should be determined by the appended claims and their legal equivalents.

### REFERENCES

Throughout the specification, citations to a number of patents, patent application publications, and literature references have been made. Each of these publications is incorporated by reference herein in its entirety. Many of the references are summarized here:
Beffert, U., Danik, M., Krzywkowski, P., Ramassamy, C., Berrada, F., and Poirier, J. (1998) The neurobiology of apolipoproteins and their receptors in the CNS and Alzheimer's disease. Brain Res Brain Res Rev 27:119-42.
Blass, J. P., and Zemcov, A. (1984) Alzheimer's disease. A metabolic systems degeneration? Neurochem Pathol 2:103-14.
Craft, S., Newcomer, J., Kanne, S., Dagogo-Jack, S., Cryer, P., Sheline, Y., Luby, J., Dagogo-Jack, A., and Alderson, A. (1996) Memory improvement following induced hyperinsulinemia in Alzheimer's disease. Neurobiol Aging 17:123-30.
Corbo, R. M. and Sacchi, R. (1999) Apoliporpotein E (APOE) allele distribution in the world. Is APOE*4 a ‘thrifty’ allele. Ann Hum Genet 63:301-10.
Davis, J. N., and Chisholm, J. C. (1999). Alois Alzheimer and the amyloid debate. Nature 400:810.
Edmond, J. (1992) Energy metabolism in developing brain cells. Can J Physiol Pharmacol 70:S118-29.
Evans, D. A., Funkenstein, H. H., Albert, M. S., Scherr, P. A., Cook, N. R., Chown, M., J., Hebert, L. E., Hennekens, C. H., and Taylor, J. O. (1989) Prevalence of Alzheimer's disease in a community population of older persons. Higher than previously reported. JAMA 262:2551-6.
Finch, C. E., and Cohen, D. M. (1997) Aging, metabolism, and Alzheimer disease: review and hypotheses. Exp Neurol 143:82-102.
Frolich, L., Blum-Degen, D., Bernstein, H. G., Engelsberger, S., Humrich, J., Laufer, S., Muschner, D., Thalheimer, A., Turk, A., Hoyer, S., Zochling, R., Boissl, K. W., Jellinger, K., and Riederer, P. (1998) Brain insulin and insulin receptors in aging and sporadic Alzheimer's disease. J Neural Transm 105:423-38.
Bullock, R. (2002). “New drugs for Alzheimer's disease and other dementias.” Br J Psychiatry 180:135-9.
Gelman, L., J. C. Fruchart, et al. (1999). “An update on the mechanisms of action of the peroxisome proliferator-activated receptors (PPARs) and their roles in inflammation and cancer.” Cell Mol Life Sci 55(6-7): 932-43.
Hertz, L., A. C. Yu, et al. (2000). “Neuronal-astrocytic and cytosolic-mitochondrial metabolite trafficking during brain activation, hyperammonemia and energy deprivation.” Neurochem Int 37(2-3): 83-102.
Schoonjans, K., J. Peinado-Onsurbe, et al. (1999). “3-Hydroxy-3-methylglutaryl CoA reductase inhibitors reduce serum triglyceride levels through modulation of apolipoprotein C-III and lipoprotein lipase.” FEBS Lett 452(3): 160-4.
Selkoe, D. J. (2001). “Alzheimer's disease: genes, proteins, and therapy.” Physiol Rev 81(2): 741-66.
Staels, B., J. Dallongeville, et al. (1998). “Mechanism of action of fibrates on lipid and lipoprotein metabolism.” Circulation 98(19): 2088-93.
Gregg, R. E., Zech, L. A., Schaefer, E. J., Stark, D., Wilson, D., and Brewer, H. B. Jr. (1986). Abnormal in vivo metabolism of apolipoprotein E4 in humans. J Clin Invest 78:815-21.
Goodman, L. S., Limbird, L. E., Milinoff, P. B., Gilman, A. G., and Hardman, J. G. (editors). (1996). The Pharmacological Basis of Therapeutics, 9.sup.th Ed., McGraw-Hill.
Hall K., Gureje O., Gao S., Ogunniyi A., Hui S. L., Baiyewu O., Unverzagt F. W., Oluwole S., Hendrie H. C. (1998) Risk factors and Alzheimer's disease: a comparative study of two communities. Aust N Z J Psychiatry 32:698-706.
Hamosh, M. (1990) In: Lingual and Gastric Lipases: Their role in fat digestion. CRC press, Boca Raton, Fla.
Hanlon C. S., and Rubinsztein D. C. (1995) Arginine residues at codons 112 and 158 in the apolipoprotein E gene correspond to the ancestral state in humans. Atherosclerosis 112:85-90.
Hasselbalch, S. G., Madsen, P. L., Hageman, L. P., Olsen, K. S., Justesen, N., Holm, S., and Paulson, O. B. (1996) Changes in cerebral blood flow and carbohydrate metabolism during acute hyperketonemia. Am J Physiol 270:E746-51.
Hertz, L., A. C. Yu, et al. (2000). “Neuronal-astrocytic and cytosolic-mitochondrial metabolite trafficking during brain activation, hyperammonemia and energy deprivation.” Neurochem Int 37(2-3): 83-102.
Hoyer, S. (1998) Is sporadic Alzheimer disease the brain type of non-insulin dependent diabetes mellitus? A challenging hypothesis. J Neural Transm 105:415-22.
Hoyer, S. (1992) Oxidative energy metabolism in Alzheimer brain. Studies in early-onset and late-onset cases. Mol Chem Neuropathol 16:207-24.
Jolles, J., Bothmer, J., Markerink, M., and Ravid, R. (1992) Phosphatidylinositol kinase is reduced in Alzheimer's disease. J Neurochem 58:2326-9.
Kolanowski, J., Young, J. B., and Landsberg L. (1994) Stimulatory influence of D(−)3-hydroxybutyrate feeding on sympathetic nervous system activity in the rat. Metabolism 43:180-5.
Klivenyi, P., Ferrante, R. J., Matthews, R. T., Bogdanov, M. B., Klein, A. M. Andreassen, O. A., Mueller, G., Wermer, M., Kaddurah-Daouk, R., and Beal, M. F. (1999) Neuroprotective effects of creatine in a transgenic animal model of amyotrophic lateral sclerosis. Nat. Med. 5:347-50.
Koo, E. H., Lansbury, P. T., Jr., and Kelly, J. W. (1999) Amyloid diseases: abnormal protein aggregation in neurodegeneration. Proc Natl Acad Sci USA. 96:9989-90.
Knouff, C., Hinsdale, M. E., Mezdour, H., Altenburg, M. K., Watanabe, M., Quarfordt, S. H., Sullivan, P. M., and Maeda, N. (1999) Apo E structure determines VLDL clearance and atherosclerosis risk in mice. J Clin Invest 103:1579-86.
Lannert, H., and Hoyer, S. (1998) Intracerebroventricular administration of streptozotocin causes long-term diminutions in learning and memory abilities and in cerebral energy metabolism in adult rats. Behav Neurosci 112:199-208.
Loktionov A., Vorster H., O'Neil I. K., Nell T., Bingham S. A., Runswick S. A., Cummings J. H. (1999) Apolipoprotein E and methylenetetrahydrofolate reductase genetic polymorphisms in relation to other risk factors for cardiovascular disease in UK Caucasians and Black South Africans. Atherosclerosis 145:125-35.
Mattson, M. P. (1998). Experimental models of Alzheimer's Disease. Science and Medicine March/April:16-25.
McKhann, G., Drachman, D., Folstein, M., Katzman, R., Price, D., and Stadlan, E. M. (1984). Clinical diagnosis of Alzheimer's disease: report of the NINCDS-ADRDA Work Group under the auspices of Department of Health and Human Services Task Force on Alzheimer's Disease. Neurology 34:939-44.
Meier-Ruge, W., Bertoni-Freddari, C., and Iwangoff, P. (1994) Changes in brain glucose metabolism as a key to the pathogenesis of Alzheimer's disease. Gerontology 40:246-52.
Messier, C., and Gagnon, M. (1996) Glucose regulation and cognitive functions: relation to Alzheimer's disease and diabetes. Behav Brain Res 75:1-11.
Neve, R. L., and Robakis, N. K. (1998) Alzheimer's disease: a re-examination of the amyloid hypothesis. Trends Neurosci 21:15-9.
Nishimura, M., Yu, G., and St George-Hyslop, P. H. (1999) Biology of presenilins as causative molecules for Alzheimer disease. Clin Genet 55:219-25.
Odle, J. (1997) New insights into the utilization of medium-chain triglycerides by the neonate: Observations from a pig model. J Nutr. 127:1061-7.
Reiman, E. M., Caselli, R. J., Yun, L. S., Chen, K., Bandy, D., Minoshima, S., Thibodeau, S. N., and Osborne, D. (1996) Preclinical evidence of Alzheimer's disease in persons homozygous for the epsilon 4 allele for apolipoprotein E. N Engl J Med 334:752-8.
Osuntokun B. O., Sahota A., Ogunniyi A. O., Gureje O., Baiyewu O., Adeyinka A., Oluwole S. O., Komolafe O., Hall K. S., Unverzagt F. W., et al (1995) Lack of an association between apolipoprotein E epsilon 4 and Alzheimer's disease in elderly Nigerians. Ann Neurol 38:463-5.
Roheim P. S., Carey M., Forte T., and Vega G. L. (1979) Apolipoproteins in human cerebrospinal fluid. Proc Natl Acad Sci USA 76:4646-9.
Schoonjans, K., J. Peinado-Onsurbe, et al. (1999). “3-Hydroxy-3-methylglutaryl CoA reductase inhibitors reduce serum triglyceride levels through modulation of apolipoprotein C-III and lipoprotein lipase.” FEBS Lett 452(3): 160-4.
Selkoe, D. J. (1994) Alzheimer's Disease: A central role for amyloid. J. Neruopathol. Exp. Neruol. 53:438-447.
Selkoe, D. J., (1999) Translating cell biology into therapeutic advances in Alzheimer's disease. Nature 399:A23-31.
Simpson, I. A., and Davies, P. (1994) Reduced glucose transporter concentrations in brains of patients with Alzheimer's disease: Ann Neurol 36:800-1.
Staels, B., J. Dallongeville, et al. (1998). “Mechanism of action of fibrates on lipid and lipoprotein metabolism.” Circulation 98(19): 2088-93.
Swaab, D. F., Lucassen, P. J., Salehi, A., Scherder, E. J., van Someren, E. J., and Verwer, R. W. (1998) Reduced neuronal activity and reactivation in Alzheimer's disease. Prog Brain Res 117:343-77.
Veech, Richard WO 98/41200. Sep. 24, 1998. Therapeutic Compositions
Veech, Richard WO 98/41201. Sep. 24, 1998. Therapeutic Compositions
Veech, Richard WO 00/15216. Mar. 23, 2000. Therapeutic Compositions (II)
Veneman, T., Mitrakou, A., Mokan, M., Cryer, P., and Gerich, J. (1994) Effect of hyperketonemia and hyperlacticacidemia on symptoms, cognitive dysfunction, and counterregulatory hormone responses during hypoglycemia in normal humans. Diabetes 43:1311-7.
Zekraoui L., Lagarde J. P., Raisonnier A., Gerard N., Aouizerate A., Lucotte G. (1997) High frequency of the apolipoprotein E*4 allele in African pygmies and most of the African populations in sub-Saharan Africa. Hum Biol 69:575-81.
Zubenko, G. S., Stiffler, J. S., Hughes, H. B., and Martinez, A. J. (1999) Reductions in brain phosphatidylinositol kinase activities in Alzheimer's disease. Biol Psychiatry 45:731-6.
Takada, A.; Ide, N.; Fukuda, T.; Miyamoto, T.; Yamagata, K.; Watanabe, J. Discotic columnar liquid crystals in oligosaccharide derivatives III. Anomeric effects on the thermo-mesomorphic properties of cellobiose octa-alkanoates (1995) Liq. Cryst. 19:441-8.
Takada, A.; Fukuda, T.; Miyamoto, T.; Yakoh, Y.; Watanabe, J (1992) Columnar liquid crystals in oligosaccharide derivatives. II. Two types of discotic columnar liquid-crystalline phase of cellobiose alkanoates Liq. Cryst. 12:337-45.
Takada, Akihiko; Itoh, Takahiro; Fukuda, Takeshi; Miyamoto, Takeaki; (1991) Preparation of cellobiose octa(n-alkanoate)s and their thermal properties Bull. Inst. Chem. Res., 69:77-83.
Jandacek, Ronald J.; Webb, Marjorie R., Physical properties of pure sucrose octaesters (1978) Chem. Phys. Lipids 22:163-76.

1. A compound of the formula: 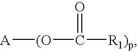
wherein A represents a saccharide moiety, p is the number of free hydroxyl groups on the saccharide moiety A, and R₁ is independently selected from a fatty acid residue having 5-12 carbons in the carbon backbone (C5 to C12 fatty acids) esterified to the saccharide, a saturated fatty acid residue having 5-12 carbons in the carbon backbone (C5 to C12 fatty acids) esterified to the saccharide, an unsaturated fatty acid residue having 5-12 carbons in the carbon backbone (C5 to C12 fatty acids) esterified to the saccharide, and derivatives of any of the foregoing, and wherein the compound is not described in Takada, et al., 1991 nor Jandacek & Webb, 1978.

2. The compound of claim 1, wherein R₁ comprises C₈ fatty acid residues.

3. The compound of claim 1 having the structure

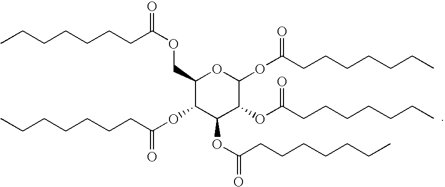

4. The compound of claim 1 having the structure

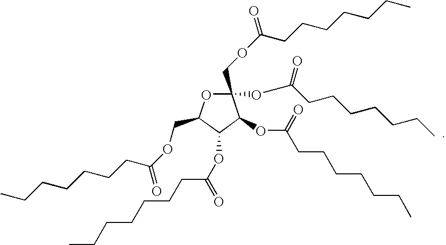

5. A compound of the formula: 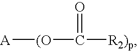
wherein R₂ is independently selected from the group consisting of R₁, an essential fatty acid esterified to the saccharide, β-hydroxybutyrate esterified to the saccharide, acetoacetate esterified to the saccharide, compound 5 esterified to the saccharide, and compound 6 esterified to the saccharide, and wherein the compound is not described in Takada, et al., 1991 nor Jandacek & Webb, 1978.

6. The compound of claim 5, wherein R₂ is either acetoacetate esterified to the saccharide or β-hydroxybutyrate esterified to the saccharide

7. The compound of claim 6, wherein the ratio of β-hydroxybutyrate R₂ groups to acetoacetate R₂ groups range from 3:2 to 4:1.

8. The compound of claim 7, wherein the ratio of β-hydroxybutyrate R₂ groups to acetoacetate R₂ groups is 3:1.

9. A mixture of a first compound of claim 6 and a second compound of claim 6, wherein the first compound R₂ group is β-hydroxybutyrate; and the second compound R₂ group is acetoacetate, and wherein the first compound and the second compound are present in a ratio ranging from 3:2 to 4:1.

10. The compound of claim 9, wherein the first compound and the second compound are present in a ratio of 3:1.

11. A pharmaceutical composition, comprising a TCA cycle intermediate and a compound selected from the group consisting of a compound of the formula 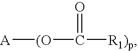
wherein A represents a saccharide moiety, p is the number of free hydroxyl groups on the saccharide moiety A, and R₁ is independently selected from a fatty acid residue having 5-12 carbons in the carbon backbone (C5 to C12 fatty acids) esterified to the saccharide, a saturated fatty acid residue having 5-12 carbons in the carbon backbone (C5 to C12 fatty acids) esterified to the saccharide, an unsaturated fatty acid residue having 5-12 carbons in the carbon backbone (C5 to C12 fatty acids) esterified to the saccharide, and derivatives of any of the foregoing; and a compound of the formula: 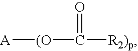
wherein R₂ is independently selected from the group consisting of R₁, an essential fatty acid esterified to the saccharide, β-hydroxybutyrate esterified to the saccharide, acetoacetate esterified to the saccharide, compound 5 esterified to the saccharide, and compound 6 esterified to the saccharide.

12. The pharmaceutical composition according to claim 11, wherein the TCA cycle intermediate is selected from a group consisting of citric acid, aconitic acid, isocitric acid, α-ketoglutaric acid, succinic acid, fumaric acid, malic acid, oxaloacetic acid, and mixtures thereof.

13. The pharmaceutical composition according to claim 12, wherein the precursor of a TCA cycle intermediate is a compound which, upon administration to a human being or animal, is converted in vivo to form a TCA cycle intermediate.

14. The pharmaceutical composition according to claim 12, wherein the precursor is selected from a group consisting of 2-keto-4-hydroxypropanol, 2,4-dihydroxybutanol, 2-keto-4-hydroxybutanol, 2,4-dihydroxybutyric acid, 2-keto-4-hydroxybutyric acid, aspartates, mono- and di-alkyl oxaloacetates, pyruvate, and glucose-6-phosphate.

15-38. (canceled)
